(19) United States
(12) Patent Application Publication
(10) Pub. No.: US 2011/0015712 A1
(43) Pub. Date: Jan. 20, 2011

(54) LIVING BODY ELECTRICITY NEEDLE

(76) Inventor: Kwan Ho Mok, Gyeonggi-do (KR)

Correspondence Address: IPLA P.A., 3550 WILSHIRE BLVD., 17TH FLOOR, LOS ANGELES, CA, 90010, US

(21) Appl. No.: 12/667,652

(22) PCT Filed: Jul. 4, 2007
(86) PCT No.: PCT/KR2008/003973

§ 371 Date: Jan. 4, 2010

(30) Foreign Application Priority Data
Jul. 4, 2007 (KR) 10-2007-0066960
Nov. 6, 2007 (KR) 10-2007-0112462

(51) Int. Cl. A61N 1/05; A61H 39/08

(52) U.S. Cl. 607/116

(57) ABSTRACT

A disposable electricity needle is disclosed, which includes a positive pole metal member which is wound on an upper outer side of a needle body having a certain length; a partition member which is positioned at a lower side of the positive pole metal member and is installed in an outer surface of the needle body and contains an electrolyte material; and a negative pole metal member which is wound on an outer surface of the partition member.

### TECHNICAL FIELD

The present invention relates to a living body electricity needle used in a meridian pathway of a human being body, and in particular to a living body electricity needle which makes a body energy flow smooth as a living body current flows in safe by means of a needle.

### BACKGROUND ART

The body of a human being receives part of electricity, and a living body current flows in a human being body. This bioelectricity is found by Gavanic. It is known that a human being at birth has about 5-6 voltages, and an adult has less than 2.5 voltages.

When a certain abnormal condition occurs in a living body, a disease occurs. The bioelectricity is applied in various ways such as in a needle therapy of an oriental medical field and a new clinical treatment of a modern science.

The needle therapy in an oriental medical field consists of an acupuncture spot and a meridian pathway. The reliabilities of the acupuncture spot and meridian pathway are unclear so far, but its anatomical truth of a meridian pathway is known by Korean scientists.

The team lead by a professor Kim, Hyun-won of Biochemistry department of Yeonsei Wonju medical school has succeeded to confirm the reality of the meridian pathway which has been known just as an energy pathway.

The team lead by a professor Kim, Hyun-won of Biochemistry department of Yeonsei Wonju medical school photographed the meridian pathways in a skin of a rabbit as well as an inner and outer side of a blood vessel, peritoneum, internal organ surface or something.

FIGS. 12 through 15 show the photos of meridian pathways.

The needle used in a needle therapy with respect to meridian pathways is formed of a traditional needle and an electric needle which is used in a therapy method with respect to a living body current.

The electricity needle is disclosed as Berlioz of France in 1816 as he treated neuralgia using a needle therapy with current, and he treated gout and neuron systems by applying an electricity needle. The foundation of the electricity needle therapy was achieved by E.A Golden in 1921 in England. It is further studied in China, Japan, Germany and other countries.

In particular, as current is continuously supplied to a blood spot by using an electricity needle, dendritic cells formed around a blood spot are decomposed and excreted through an excretion pipe for thereby keeping a healthy body. It can be also used for releasing quick or chronicle pains after baby delivery.

### DISCLOSURE

### Technical Problem

However, the electricity used for an electricity needle is supplied from an AC current. It is needed to flow a very small amount of electricity in a human being body. If an electric apparatus fails to normally operate, a relatively high current might flow in a human being body, which might lead to an electric shock. In worse case, a patient might be dead by means of electric shock.

The same applicant as the present application invented and filed an electricity needle safer and simple, which is disclosed in Korean patent registration number 10-0724106.

However, the above patent has problems in portability.

### Technical Solution

Accordingly, it is an object of the present invention to provide a living body electricity needle which overcomes the problems found in the conventional art and is equipped with a reliable portability and is manufactured at a lower cost.

To achieve the above objects, there is provided a disposable electricity needle in which a positive pole metal member and a negative pole metal member are installed on an upper side of a needle body, and a partition member having an electrolyte is installed between the positive and negative pole metal members.

The positive pole metal member is one selected from the group consisting of Au, Pt, Ag, Cu, Pb, Sn, and Ni.

The negative pole metal member is one selected from the group consisting of Ca, K, Na, Mg, Al, Zn, and Fe.

The partition members 6, 6′, 6″ is one selected from the group consisting of sponge, pulp fiber and film sheet which contain water, sulphuric acid or salt which are electrolyte materials.

### Advantageous Effects

The portability and movement can be enhanced in the living body electricity needle according to the present invention, and the products of the present invention can be manufactured at lower prices.

### DESCRIPTION OF DRAWINGS

FIG. 1 is a view illustrating a living body electricity needle according to a first embodiment of the present invention.

FIG. 2 is a view illustrating a living body electricity needle according to a second embodiment of the present invention.

FIG. 3 is a view illustrating a living body electricity needle according to a third embodiment of the present invention.

FIG. 4 is a cross sectional view illustrating a living body electricity needle according to a third embodiment of the present invention.

FIG. 5 is a view illustrating a living body electricity needle according to a fourth embodiment of the present invention.

FIG. 6 is a cross sectional view taken along line I-I while illustrating a living body electricity needle according to a fourth embodiment of the present invention.

FIG. 7 is a view illustrating a living body electricity needle according to a 5-1 embodiment of the present invention.

FIG. 8 is a cross sectional view taken along line I-I while illustrating a living body electricity needle according to a 5-1 embodiment of the present invention.

FIG. 9 is a view illustrating a living body electricity needle according to a 5-2 embodiment of the present invention.

FIG. 10 is a cross sectional view taken along line I-I while illustrating a living body electricity needle according to a 5-2 embodiment of the present invention.

FIG. 11 is a view illustrating a living body electricity needle according to a sixth embodiment of the present invention.

FIGS. 12 through 15 are photos taken with respect to meridian pathways.

### BEST MODE

There is provided a disposable electricity needle, comprising a positive pole metal member 4 which is wound on an upper outer side of a needle body 2 having a certain length; a partition member 6 which is positioned at a lower side of the positive pole metal member 4 and is installed in an outer surface of the needle body 2 and contains an electrolyte material; and a negative pole metal member 8 which is wound on an outer surface of the partition member 6.

### Mode for Invention

The preferred embodiments of the present invention will be described with reference to the accompanying drawings.

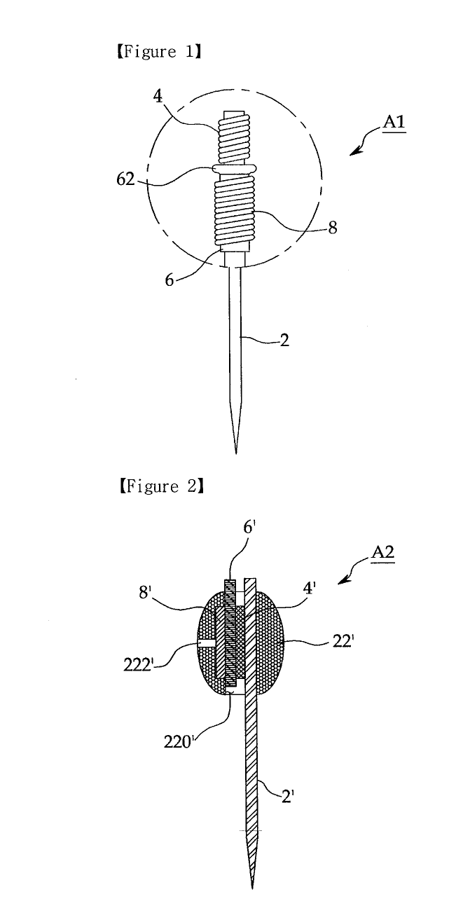

FIG. 1 is a view illustrating a living body electricity needle according to a first embodiment of the present invention.

### First Embodiment

As shown in FIG. 1, the first embodiment Al comprises a positive pole metal member 4 wound on an outer surface of a top of a needle body 2 having a certain length, a partition member 6 which is positioned in a lower side of the positive pole metal member 4 and installed in an outer surface of the needle body 2, and a negative pole metal member 8 which is wound on an outer surface of the partition member 6.

The positive and negative metal members 4 and 8 might change their installation positions with each other.

The needle body 2 is made of a conductive metallic material and has a certain length in a rod shape with its lower end being formed of a sharp tip, with its upper end being engaged with a coil or rod body for thereby forming a head part having a thick diameter.

The positive pole metal member 4 is wound on an outer side of the head part of the needle body in a coil shape and is made of one selected from the group consisting of Au, Pt, Ag, Cu, Pb, Sn and Ni. In the present embodiment, Cu is preferably used.

The partition member 6 is formed of a cylindrical hollow passage, so the needle body is inserted into the same. A circular shoulder 62 is formed in its upper end for thereby defining a boundary between the positive and negative metal members 4 and 8, and it is made of one selected from the group consisting of sponge, pulp fiber and film sheet which contain electrolyte water, sulphuric acid, or salt component.

Since the partition member 6 has the same composition in the following other embodiments of the present invention, the descriptions of the same will be omitted.

The negative metal member 8 is wound on an outer surface of the partition member 6 and is made of one selected from the group consisting of Ca, K, Mg, Al, Zn, and Fe. In the present embodiment, Zn is preferably used.

The positive and negative pole metal members 4 and 8 might change their installation positions with each other.

Since the positive and negative metal members 4 and 8 has the same composition in the following other embodiments of the present invention, the descriptions of the same will be omitted.

When the needle of the first embodiment Al is treated on a meridian pathway, and water is wetted on its upper side, the positive pole metal member 4 of the inner side has positive electricity, and the negative pole meta member 8 of the outer side has negative electricity, so current flows between the positive and negative pole metal members 4 and 8 by means of an electric potential difference.

So, the low level current generated through the needle advantageously stimulates the meridian pathways for thereby smoothing the flow of blood.

### Second Embodiment

As shown in FIG. 2, the second embodiment A2 comprises a handle 22′ having an insertion hole 220′ in its inner side, and a needle body ′ of which its upper end is inserted into an insertion hole 220′. A positive pole meal member 4′ directly contacting with an outer surface of a needle body 2″ is installed in one side of an inner surface of an insertion hole 220″, and a negative pole metal member 8′ is installed in the other side of an inner surface of an insertion hole 220′ at a space from the outer surface of a needle body 2′, and a partition member 6′ is installed between the positive and negative pole metal members 4′ and 8′.

The needle body 2′ is vertically inserted into the insertion hole 220′ of the handle 22′, and the positive pole metal member 4′ is inserted while contacting with the needle body 2′, and the partition member 6′ is inserted while contacting with the positive pole metal member 4′. The negative pole metal member 8′ is inserted while contacting with the partition member 6′.

Here, the positive and negative pole metal members 4′ and 8′ might change their installation positions with each other.

A through hole 222″ is formed in a side of the handle 22′ while communicating with the insertion hole 220′ of the inner side, and electrolyte substance is absorbed through the through hole 222″.

The needle body 2′ is made of a conductive metallic material, and part of the same is exposed from the upper side of the handle 22′.

The partition member 6′ is embedded in the insertion hole 220′ and is protruded from the upper side of the handle 22′.

In the second embodiment of the present invention, when water is wetted on the needle body 2′ protruded from the upper side of the handle 22′, the positive pole metal member 4′ has a positive electricity, and the negative pole metal member 8′ of the outer side has negative electricity, and current flows in the needle body 2′ by means of an electric potential difference between the positive and negative pole metal members 4′ and 8′.

When the needle body 2′ is treated on an acupuncture stop, a low level current stimulates the meridian pathway for thereby smoothing the flow of blood.

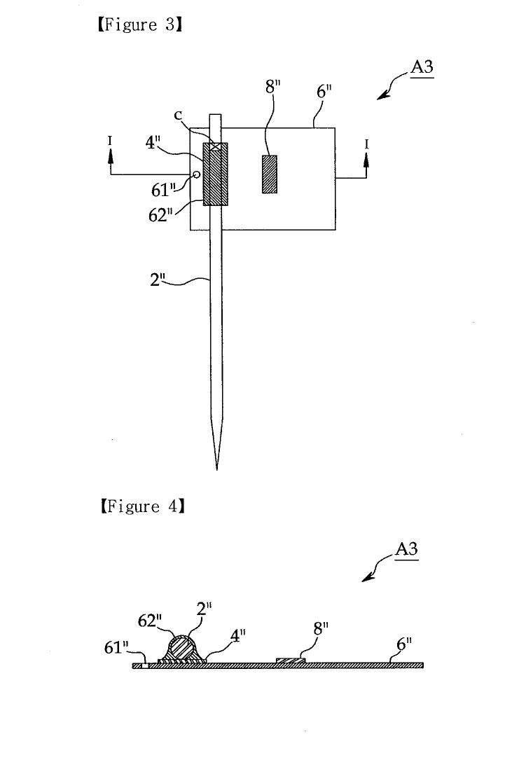

FIG. 3 is a view illustrating a living body electricity needle according to a third embodiment of the present invention. FIG. 4 is a cross sectional view illustrating a living body electricity needle according to a third embodiment of the present invention.

### Third Embodiment

As shown in FIGS. 3 and 4, the third embodiment A3 of the present invention comprises a film sheet 6″ having a positive pole metal member 4″ formed by silk-printing on an inner side with a thin plate, and a negative pole metal member 8″ spaced apart from the positive pole metal member 4″ and silk-printed with a thin plate, a needle body 2″ which is attached to the film sheet 6″ as its lower outer side is attached (c) to the positive pole metal member 4″, and an electrolyte membrane 62″ coated on an upper side of the needle body 2″ for covering the positive pole metal member 4″.

As shown in FIG. 3, a certain through hole 61″ is formed in one end of the film sheet 6″, namely, in the left most side, so moisture is inputted through the same and is transferred to the positive pole metal member 4″.

So, when the film sheet 6″ is wound from the left most side and covers the needle body 2″, the positive pole metal member 4″ contacts with one side of the needle body 2″, and the negative pole metal member 8″ is arranged on an outer side of the needle body 2″ between the film sheet 6″ having the through hole 61″ in its left side and the electrolyte membrane 62″.

In addition, the needle body 2″ is installed with its upper side being protruded from the upper side of the film sheet 6″.

When the film sheet 6″ is wound on the negative pole metal member 8″, it is formed in a size smaller than the positive pole metal member 4″ for preventing contacts with the adhered portion(c) of the positive pole metal member 4″ contacting with the needle body 2″.

In the third embodiment of the present invention, when the upper side of the needle body 2″ is wetted with water, the negative pole metal member 8″ has negative electricity, and the positive pole metal member 4″ has positive electricity, and the current flows in the needle body 2″ by means of an electric potential difference between the positive and negative pole metal members 4″ and 8″.

When the needle body 2″ is treated on an acupuncture spot, a low level current stimulates the meridian pathway for thereby smoothing the flow of blood.

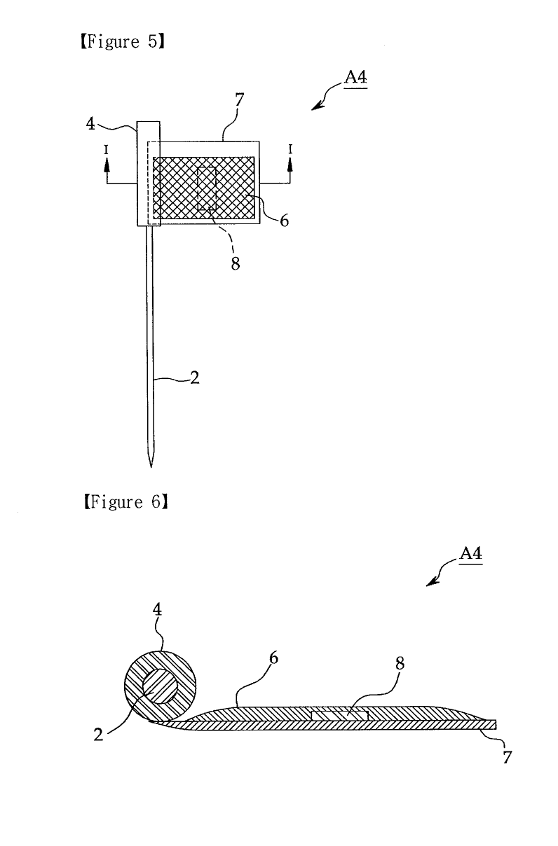

FIG. 5 is a view illustrating a living body electricity needle according to a fourth embodiment of the present invention. FIG. 6 is a cross sectional view taken along line I-I while illustrating a living body electricity needle according to a fourth embodiment of the present invention.

### Fourth Embodiment

As shown in FIGS. 5 and 6, the fourth embodiment A4 of the present invention comprises a positive pole metal member 4 coated on an outer side of a head part of a needle body 2 having a certain length, a finishing material 7 which is wound on an outer side of the positive pole metal member 4, a negative pole metal member 8 which is silk-printed on an inner surface of the finishing material 7, and a partition member 65 which is attached on an upper surface of the negative pole metal member 8.

The finishing material 4 is formed in a rectangular tape shape, and as shown in FIG. 6, its left most end is fixed in a lower side of the positive pole metal member 4 of the needle body 2.

At this time, the needle body 2 is protruded from the upper side of the finishing material 7.

Here, the positive and negative pole meal members 4 and 8 might change their installation positions with each other. This construction is described in the first embodiment, so its description is omitted.

The partition member 6 is made of a fiber or a pulp sheet which is capable of well absorbing moisture. It contains an electrolyte material.

When the finishing material 7 is wound from a portion fixed in the positive pole metal member 4, the negative pole metal member 8 is disposed between the partition members 6.

In the fourth embodiment A4 of the present invention, when water is wetted on the upper side of the needle body 2, the positive pole metal member 4 has positive electricity, and the negative pole metal member 8 has negative electricity, so the current flows in the needle body due to an electric potential difference between the positive and negative pole metal members 4 and 8.

So, when the needle body 2 is treated on the acupuncture spot, a low level current stimulated the meridian pathway for smoothing the flow of blood.

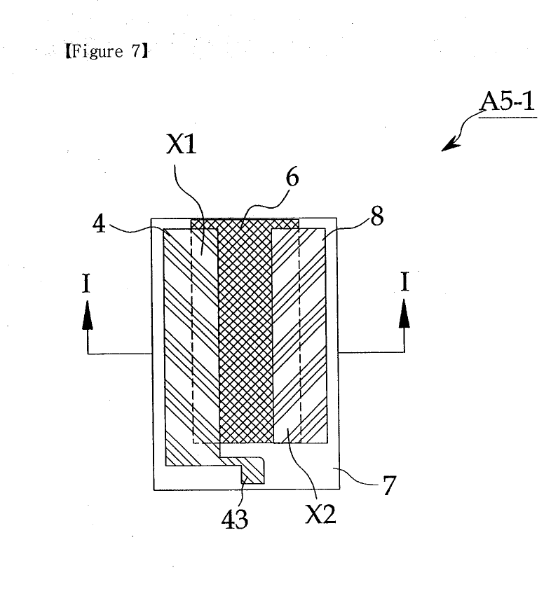

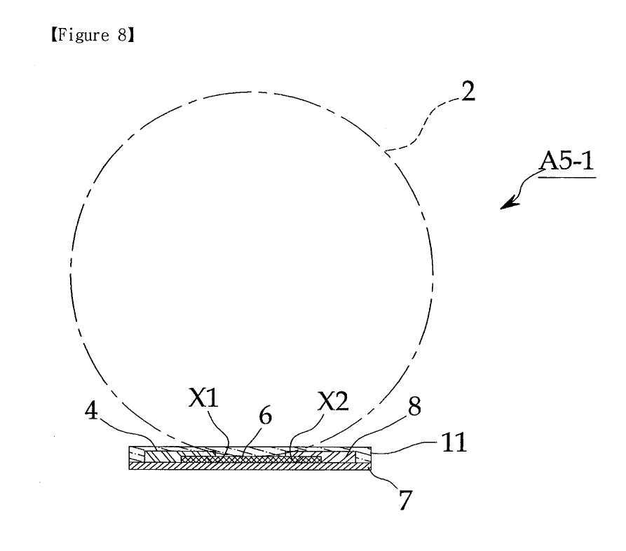

FIG. 7 is a view illustrating a living body electricity needle according to a 5-1 embodiment of the present invention. FIG. 8 is a cross sectional view taken along line I-I while illustrating a living body electricity needle according to a 5-1 embodiment of the present invention.

### 5-1 Embodiment

As shown in FIGS. 7 and 8, the 5-1 embodiment of the present invention is characterized that in the living body electricity needle formed of a needle body 2 having a certain length, and an electricity generation unit (e) attached to the needle body 2 for generating electricity, the electricity generation unit (2) is formed in a stick shape, and comprises a longitudinal film sheet 7 coated with an adhesive on its upper surface, a partition member 6 which is attached in a thin plate shape on an upper center of the film sheet 7, a positive pole metal member 4 in which a metallic powder silk is printed on one upper side of the film sheet 7 and is overlapped on an upper side of the partition member 6, with an extension 43 being formed at its one end up to a center of the film sheet 7, a negative pole metal member 8 in which a metallic powder silk is printed on an upper side of the other side of the film sheet 7 and is overlapped on an upper side of the other side of the partition member 6, and is spaced apart from the positive pole metal member 4, and a release paper 11 which is coated on the whole upper sides of the film sheet 7, the partition member 6, the positive pole meta member 4 and the negative pole metal member 8.

The electricity generation unit (e) is formed in a stick shape and is attachable to any kinds of needles and is disposable.

The partition member 6 is made of a fiber material or a pulp sheet which is capable of easily absorbing moisture and is adhered by means of a conductive adhesive.

The positive pole metal member 4 is formed on an upper side of the film sheet 7, and is overlapped (X1) on one side of the partition member 6, with its one end being extended up to a center of the film sheet for thereby forming an extension part 43 directly contacting with the needle body.

The negative pole metal member 8 is paced apart from the positive pole metal member 4 and is formed on an upper side of the other side of the film sheet 7 whole overlapping with the other side of the partition member 6.

The positive and negative pole metal members 4 and 8 are not limited to the metallic powders, and are obviously metallic solids.

The release paper 11 is a plastic cover attached for protecting the partition member 6 and the film sheet 9 and should be removed before use.

As shown in FIG. 8, the release paper 11 is removed, and the head part of the needle body 2 is pushed with respect to the partition member 6, and is adhered to the film sheet 7.

The needle body 2 might be all kinds of known needles used for needle therapy.

When the upper side of the needle body 2 is wetted by water, the positive pole metal member 4 has positive electricity, and the negative pole metal member 8 has negative electricity, and the current flows in the needle body 2 by means of an electric potential difference between the positive and negative pole metal members 4 and 8.

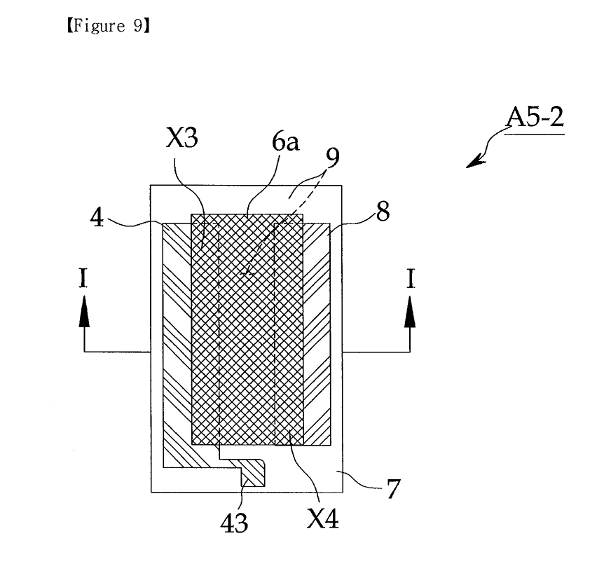

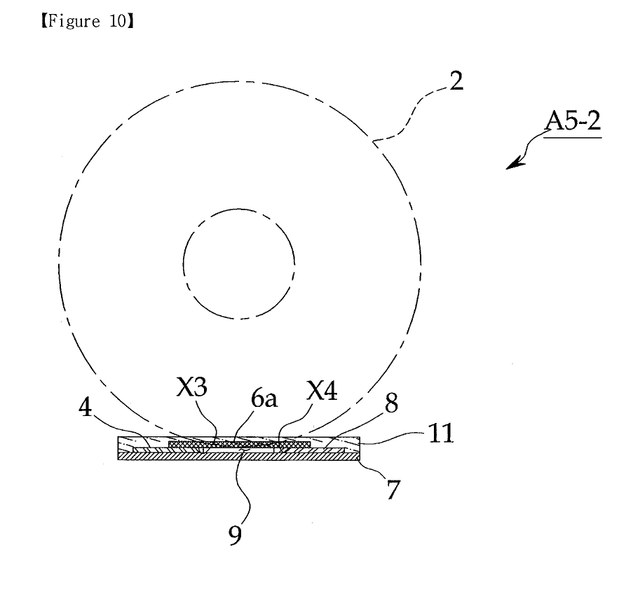

FIG. 9 is a view illustrating a living body electricity needle according to a 5-2 embodiment of the present invention. FIG. 10 is a cross sectional view taken along line I-I while illustrating a living body electricity needle according to a 5-2 embodiment of the present invention.

### 5-2 Embodiment

As shown in FIGS. 9 and 10, the 5-2 embodiment A5-2 of the present invention is characterized in that in the living body electricity needle having a needle body 2 having a certain length, and an electricity generation unit (e) attached to the needle body 2 for generating electricity, the electricity generation unit (e) is formed in a stick shape and comprises a longitudinal film sheet 7 with an upper lower side being coated with an adhesive, a positive pole metal member 4 is formed by silk-printing metallic powder on an upper side of the film sheet 7, and has an extension part 43 extended up to a center of the film sheet 7 in one end, a negative pole metal member 8 is formed by silk-printing on an upper side of the other side of the film sheet 7 with metallic powders, and is spaced apart from the positive pole metal member 4, an electrolyte layer 6a which is formed by coating an absorption material on an upper center portion of the film sheet 7 with its both sides being partially overlapped with the positive and negative pole metal members 4 and 8, and a release paper 11 which is coated on the whole portions of the film sheet 7, the electrolyte layer 6a and the positive and negative pole metal members 4 and 8.

The electricity generation unit (e) is formed in a stick shape and is attachable to any kinds of needles and is disposable.

The electrolyte layer 6a is formed by coating absorption chemical material.

A space 9 of which part can induce water is formed between the upper surface of the film sheet 7 and the electrolyte layer 6a.

Since the construction of the film sheet 7, the positive and negative pole metal members 4 and 8, and the release paper 11 is the same as the 5-1 embodiment, its description will be omitted.

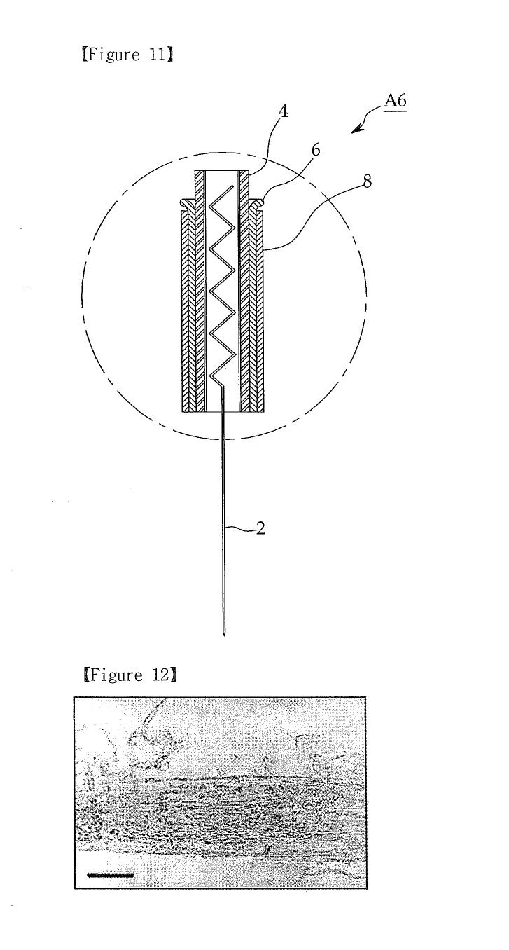

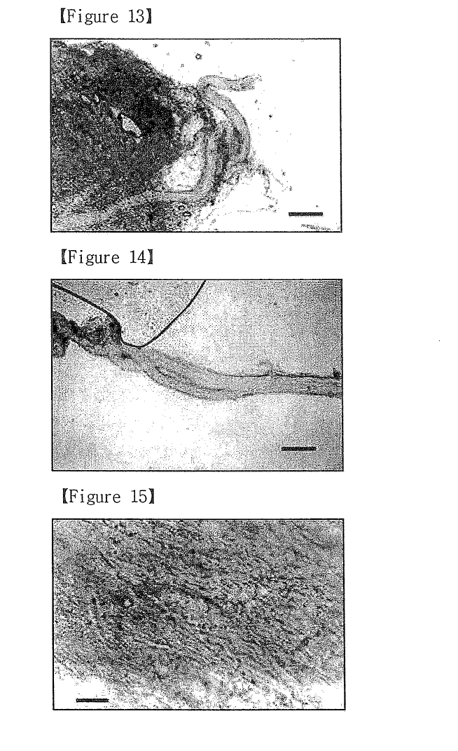

FIG. 11 is a view illustrating a living body electricity needle according to a sixth embodiment of the present invention.

### Sixth Embodiment

As shown in FIG. 11, the sixth embodiment A6 of the present invention comprises a positive pole metal member 4 coated on an outer side of a head part of a needle body 2 having a certain length, pipe shaped partition member 6 inserted into an outer side of the positive pole metal member 4, and a negative pole metal member 8 formed in a pipe shape and wound on an outer side of the partition member 6.

The needle body 2 is formed by coating a positive pole metal member 4 on an outer surface of the head part of the upper side with part of the head part being exposed to the outside.

The partition member 6 is formed in a pipe shape using paper, and preferably contains an electrolyte material.

The negative pole metal member 8 is formed in a tape shape while being wound on an outer side of the head part of the needle body 2 engaged with the partition member 6.

The positive pole metal member 4 is coated on an outer side of the head part of the needle body 2, and the pipe shaped partition member 6 is inserted into an outer side of the same, and a tape shaped negative pole metal member 8 is wound on an outer surface of the partition member 6.

When water is wetted on the upper side of the needle body 2, the partition member 6 absorbs water, and the positive pole metal member 4 has positive electricity, and the negative pole metal member 8 has negative electricity, and the current flows in the needle body 2 by means if an electric potential difference between the positive and negative pole metal members 4 and 8.

When the needle body 2 is treated on the acupuncture spot, a low level current stimulates the meridian pathway for thereby smoothing the flow of blood.

### INDUSTRIAL APPLICABILITY

The portability and movement can be enhanced in the living body electricity needle according to the present invention, and the products of the present invention can be manufactured at lower prices.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

### Sequence List Text

needle, blood, current, positive pole, negative pole

1-25. (canceled)

26. A living body electricity generation needle, comprising:
a positive pole metal member (4) which is either wound on an upper outer side of a needle body (2) having a certain length or coated on an outer side of a tip portion of the needle body (2);
a tape-shaped partition member (6) which is positioned at a lower side of the positive pole metal member 4 and is installed in an outer surface of the needle body (2) and contains an electrolyte material or inserted into an outer side of the positive pole metal member (4); and
a negative pole metal member (8) which is wound on an outer surface of the partition member (6).

27. The needle of claim 1, further comprising a finishing material (7) which is wound on an outer side of the positive pole metal member (4), with the negative pole metal member (8) being silk-printed on an inner surface of the finishing material (7), with the partition member (6) being attached to an upper side of the negative pole metal member (4).

28. A living body electricity generation needle, comprising:
a structure including a handle (22′) having an insertion hole (220′) in its inner side; a needle body (2′) of which upper end is inserted into the insertion hole (220′);
a positive pole metal member (4′) which directly contacts with an outer surface of the needle body (2′) in one side of an inner surface of the insertion hole (220′);
a negative pole metal member (8′) which is spaced apart from the outer surface of the needle body (2′) in the other side of an inner surface of the insertion hole (220′);
and a partition member (6′) which is installed between the positive and negative pole metal members (4′) and (8′) and contains an electrolyte material or a structure including a film sheet 6″ having a positive pole metal member (4″) formed by silk-printing on an inner side using a thin plate, and a negative pole metal member (8″) spaced apart from the positive pole metal member (4″) and formed by silk-printing with a thin plate; a needle body (2″) which is vertically installed in the film sheet (6″) with its lower outer side being adhered (c) to the positive pole metal member (4″);
and an electrolyte membrane (62″) coated on the upper sides of the positive pole metal member (4″) and the needle body (2″), respectively.

29. In a living body electricity needle which has a needle body (2) having a certain length and an electricity generation unit (e) attached to the needle body (2) for generating electricity, a living body electricity generation needle, comprising:
said electricity generation unit (e) being formed in a stick shape; and comprising:
a longitudinal film sheet (7) coated with an adhesive on its upper surface;
a partition member (6) which is attached to an upper center portion of the film sheet (7) in a thin plate shape;
a positive pole metal member (4) which is formed by silk-printing on an upper surface of the film sheet (7) with metallic powder and is overlapped on an upper side of the partition member (6) and has an extension part (43) formed at its one end and extended up to the center of the film sheet (7);
a negative pole metal member (8) which is formed by silk-printing metallic powder on an upper side of the other side of the film sheet (7) and is overlapped on an upper side of the partition member (6) and is spaced part from the positive pole metal member (4) by a certain distance; and
a release paper which is coated on the whole portions of the film sheet (7), the partition member (6), the positive pole metal member (4) and the negative pole metal member (8).

30. The needle of claim 5, wherein said electricity generation unit (e) being formed in a stick shape comprises:
a longitudinal film sheet (7) coated with an adhesive on its lower of an upper surface;
a positive pole metal member (4) which is formed by silk-printing metallic powder on an upper surface of the film sheet (7) with its end being equipped with an extension part (43) extended up to a center of the film sheet (7);
a negative pole metal member (8) which is formed by sink-printing metallic powder on an upper surface of the other side of the film sheet (7) and is spaced apart from the positive pole metal member (4) at a certain distance;
an electrolyte layer (6a) which is formed by coating an absorption material on an upper center portion of the film sheet (7) and is overlapped partially on the positive and negative pole metal members (4) and (8); and
a release paper which is coated on the whole portions of the film sheet (7), the electrolyte layer (6a), and the positive and negative pole metal members (4) and (8).

31. The needle of claim 1, wherein said positive pole metal member (4, 4′, 4″) is one selected from the group consisting of Au, Pt, Ag, Cu, Pb, Sn, and Ni.

32. The needle of claim 2, wherein said positive pole metal member (4, 4′, 4″) is one selected from the group consisting of Au, Pt, Ag, Cu, Pb, Sn, and Ni.

33. The needle of claim 1, wherein said partition members (6, 6′) is one selected from the group consisting of sponge, pulp fiber and film sheet which contain water, sulphuric acid or salt which are electrolyte materials.

34. The needle of claim 2, wherein said partition members (6, 6′) is one selected from the group consisting of sponge, pulp fiber and film sheet which contain water, sulphuric acid or salt which are electrolyte materials.

35. The needle of claim 5, wherein said partition members (6, 6′) is one selected from the group consisting of sponge, pulp fiber and film sheet which contain water, sulphuric acid or salt which are electrolyte materials.

36. The needle of claim 1, wherein said negative pole metal member (8, 8′, 8″) is one selected from the group consisting of Ca, K, Na, Mg, Al, Zn, and Fe.

37. The needle of claim 2, wherein said negative pole metal member (8, 8′, 8″)is one selected from the group consisting of Ca, K, Na, Mg, Al, Zn, and Fe.

38. The needle of claim 1, wherein said positive or negative pole metal members (4, 4′, 4″) and (8, 8′, 8″) might change their installation positions with each other.

39. The needle of claim 2, wherein said positive or negative pole metal members (4, 4′, 4″) and (8, 8′, 8″) might change their installation positions with each other.

40. The needle of claim 5, wherein said positive or negative pole metal members (4, 4′, 4″) and (8, 8′, 8″) might change their installation positions with each other.

41. The needle of claim 3, wherein a through hole (222′) is formed in one side surface of the handle (22′), so the insertion hole (220′) passes through the same, and electrolyte material is absorbed through the through hole (222′).

42. The needle of claim 3, wherein said negative pole metal member (8″) is smaller than the positive pole metal member (4″), so that the adhering portion of the positive pole metal member (4″) contacting with the needle body (2″) does not contact.

43. The needle of claim 2, wherein said finishing material (7) is formed in a rectangular tape shape, and is spaced downwards from an upper side of the needle body (2) and is fixed, so that the upper side of the needle body (2) can be visible from the outside.

44. The needle of claim 5, wherein said electrolyte layer (6a) is formed by coating absorption chemical material.

45. The needle of claim 5, wherein a space (9) is formed between the upper surface of the film sheet (7) and the electrolyte layer (6a) for inducing water.
